(19) United States
(12) Patent Application Publication
(10) Pub. No.: US 2015/0366932 A1
(43) Pub. Date: Dec. 24, 2015

(54) METHODS FOR PRODUCING DIKETOPIPERAZINES AND COMPOSITIONS CONTAINING DIKETOPIPERAZINES

(71) Applicant: AMPIO PHARMACEUTICALS, INC., Englewood, CO (US)

(72) Inventor: David BAR-OR, Englewood, CO (US)

(21) Appl. No.: 14/765,018

(22) PCT Filed: Feb. 3, 2014
(86) PCT No.: PCT/US14/14478

§ 371 Date: Jul. 31, 2015

Related U.S. Application Data
(60) Provisional application No. 61/759,922, filed on Feb. 1, 2013.

(51) Int. Cl. A61K 38/05; C12P 21/06; A61K 38/38

(52) U.S. Cl. CPC A61K 38/05; A61K 38/385; C12P 21/06

(57) ABSTRACT

Methods of making increased amounts of diketopiperazines (DKP) such as DA-DKP in pharmaceutical compositions of proteins and peptides are disclosed. The disclosure further provides methods of making a DKP, including (1) contacting albumin with an enzyme (such as a dipeptidyl peptidase N (DPP-IV)) that cleaves a pair of N-terminal amino acids from the albumin, and (2 heating the albumin under conditions effective to cause the formation of the DKP. Further, treatment of DKP- and albumin-containing streams to produce improved, higher value, DKP compositions and purified albumin compositions for therapeutic uses is also disclosed. In addition to a first therapeutic DKP composition comprising a low albumin content, a second valuable therapeutic composition is also produced characterized by a high albumin concentration.

This application claims the benefit of U.S. Provisional Application No. 61/759,922, filed Feb. 1, 2013, which is incorporated herein by reference in its entirety.

### TECHNICAL FIELD

Methods are provided for producing diketopiperazines, such as aspartate-alanine diketopiperazine (DA-DKP), including methods employing peptidases such as dipeptidyl peptidase IV (DPP-IV). Methods are also provided for making pharmaceutical compositions of proteins and peptides that increase the content of diketopiperazines in the compositions. Further, methods are provided for the treatment of diketopiperazine- and albumin-containing streams to produce diketopiperazine compositions and purified albumin compositions for therapeutic uses. In addition to a first therapeutic diketopiperazine composition comprising a low albumin content, a second therapeutic composition can be produced characterized by a high albumin concentration.

### BACKGROUND

Albumin is a soluble, monomeric, globular protein (molecular weight of about 66 kDa) and is the most abundant protein found in mammalian blood plasma, present in normal concentrations ranging from 0.03 to 0.05 grams per milliliter. Albumin serves several essential roles in the cardiovascular system including maintenance of oncotic pressure. Higher concentrations of albumin result in the expansion of blood plasma volume by shifting fluid from the intracellular spaces in the surrounding tissue, to the intravascular system. In addition, albumin serves as a transport protein for delivering steroid hormones, hemin and fatty acids. Albumin also helps to maintain blood pH and is involved in coagulation pathways.

Because of these essential functions, normal albumin concentrations in the blood stream are vital for maintaining homeostasis. Decreases or increases in blood albumin concentrations can lead to severe health issues. Low albumin concentrations in the blood, hypoalbuminaemia, can result from disease such as liver dysfunction and renal disorders, as well as from trauma, severe burns, and sepsis. Other conditions that have shown to benefit from albumin therapy include, but are not limited to, malnutrition, starvation, nephrotic syndrome, pancreatitis and peritonitis. For this reason, pasteurized albumin containing solutions are often administered in the operating room and in emergency medical care situations as resuscitative fluids.

The use of commercial human serum albumin (HSA) solutions in the critically ill is sometimes indicated for blood volume restoration in certain conditions such as burn, acute lung injury (ALI), and shock. For these patients, HSA administration is controversial, with recent evidence demonstrating at best no reduction in mortality rates in comparison with cheaper alternatives such as saline. In addition, the heterogeneity of commercial HSA solutions has been demonstrated and includes oxidation and truncation of the HSA molecule. During processing and storage of commercial HSA solutions, the protein truncation occurs at the N-terminus of the protein and results in the cleavage of the first two amino acids of HSA, Asp-Ala. Due to the unique nature of the N-terminus of HSA, this dipeptide is further converted to a cyclic dipeptide termed aspartate-alanine diketopiperazine (DA-DKP), also known as 3-methyl-2,5-diketopiperazine-6-acetic acid. DA-DKP has been found in significant quantities in commercial HSA solutions, and DA-DKP itself has immunosuppressive effects on activated PBMC and T-lymphocytes in vitro.

The mechanism of formation of DA-DKP from HSA is currently unknown, but auto-degradation of the N-terminus and/or an enzymatic reaction involving a peptidase could theoretically contribute. Dipeptidyl peptidase IV (DPP-IV), also known as adenosine deaminase complexing protein 2 or CD26 (cluster of differentiation 26), is a peptidase that preferentially cleaves Xaa-Pro and Xaa-Ala dipeptides from the N-terminus of proteins. DPP-IV activity has been reported on the cell surface of immune and endothelial cells as well as in blood serum as a soluble form. The main function of DPP-IV is thought to be the modification of biologically active peptides, cytokines, and other cell surface proteins for the purpose of regulating the immune response and cell differentiation. Also, a novel mechanism has been elucidated involving the DPP-IV-mediated degradation of the extracellular matrix (ECM) leading to the invasion of endothelial cells into collagenous matrices.

DKPs have been shown to have their own unique therapeutic uses, including the potential to treat human autoimmune disorders. For example, DA-DKP has been shown to have significant immunosuppressive effects on activated peripheral blood mononuclear cells and T-lymphocytes. Further disclosures regarding HSA, DA-DKP and therapeutic treatments associated therewith can be found in U.S. Pat. No. 6,555,543; U.S. Pat. No. 7,732,403 and U.S. Patent Publication No. US 2013-0090292 A1, all of which are incorporated herein by reference in their entirety.

Thus, given the potential therapeutic uses of DKPs for treating human autoimmune and other disorders and the importance of albumin for treating hypoalbuminaemia, hypovolemia and a variety of other disorders, there is a need for both high quality therapeutic DKP compositions and high quality albumin resuscitative fluids. This disclosure relates to methods for producing both of these therapeutic compositions in efficient, high-yield processes.

### SUMMARY

Administration of commercial human serum albumin (HSA) is potentially indicated in patients such as multi-trauma patients. Due to its heterogeneous nature, other components can contribute to the therapeutic effect of commercial HSA, such as proteases. One such protease, dipeptidyl peptidase IV (DPP-IV), can release a known immunomodulatory molecule from the N-terminus of albumin, aspartate-alanine diketopiperazine (DA-DKP). Commercial HSA solutions prepared, e.g., by Cohn fractionation were shown to have DPP-IV activity.

One aspect of the present disclosure is the production of DKPs, such as DA-DKP. In an embodiment, DA-DKP is produced from albumin in the presence of DPP-IV. In an embodiment, the DPP-IV is endogenous, such as in human plasma or HSA. In an embodiment, the plasma or HSA is heated. While not wishing to be bound by any theory, it is believed the heating may increase the concentration of DPP-IV by raising the temperature of the solution closer to an optimum temperature for DPP-IV activity and/or by thermal degradation.

Another aspect of the present disclosure is a method for treating a feed stream comprising albumin and DKP, such as DA-DKP, to produce compositions, the method comprising processing the feed stream to produce a first albumin-lean stream and a first albumin-rich stream, wherein the first albumin-lean stream comprises a first portion of the DKP present in the feed stream, and the first albumin-rich stream comprises a second portion of the DKP present in the feed stream. The first albumin-rich stream is reacted in order to produce additional DKP, resulting in a reaction stream comprising albumin and DKP. The reaction stream is processed to produce a second albumin-lean stream and a second albumin-rich stream, wherein the second albumin-lean stream comprises a portion of the DKP present in the reaction stream, and the second albumin-rich stream comprises a second portion of the DKP present in the reaction stream.

In some embodiments of the present disclosure, the albumin-rich streams produced can have therapeutic value, including but not limited to, effectiveness in treating hypoalbuminaemia and hypovolemia. In some embodiments of the present disclosure, the albumin-lean, DKP-containing streams produced can have therapeutic value, including but not limited to, effectiveness in treating inflammatory conditions.

A further aspect of the present disclosure, is a method for treating a feed stream comprising albumin and DKP to produce therapeutic compositions, as described above, further comprising an analyzing step, wherein the analyzing step comprises analyzing an albumin-rich stream to yield at least one metric, comparing the at least one metric to at least one reference value, wherein when the at least one metric is greater or less than the reference value, the reacting and processing steps are repeated until the at least one metric of a subsequent albumin-rich stream is equal to or less or greater than the at least one reference value. For example, the metric can be the amount of albumin in the stream or the amount of DA-DKP.

A further aspect of the present disclosure is a composition comprising DKP that contains less than about the concentration of albumin in commercial human serum albumin (“HSA”) preparations, which is about 50 grams albumin per liter of HSA (g/L) in a 5 wt % albumin solution or about 250 g/L in a 25 wt % albumin solution. In some embodiments of the present disclosure, the concentration of albumin in the DKP-containing composition can be less than about 250 g/L, less than about 200 g/L, less than about 100 g/L, less than about 50 g/L, less than about 40 g/L, less than about 30 g/L, less than about 20 g/L, less than about 10 g/L, less than about 5 g/L, less than about 4 g/L, less than about 3 g/L, less than about 2 g/L, less than about 1 g/L, less than about 0.9 g/L, less than about 0.8 g/L, less than about 0.7 g/L, less than about 0.6 g/L, less than about 0.5 g/L, less than about 0.4 g/L, less than about 0.3 g/L, less than about 0.2 g/L, less than about 0.1 g/L, less than about 0.09 g/L, less than about 0.08 g/L, less than about 0.07 g/L, less than about 0.06 g/L, less than about 0.05 g/L, less than about 0.04 g/L, less than about 0.03 g/L, less than about 0.02 g/L, less than about 0.01 g/L, less than about 0.009 g/L, less than about 0.008 g/L, less than about 0.007 g/L, less than about 0.006 g/L, or less than about 0.005 g/L. In still further embodiments of the present disclosure, the concentration of albumin in the DKP-containing composition can be about zero g/L, or non-detectable amounts.

In some embodiments of the present disclosure, compositions comprising DKP can have therapeutic value, including but not limited to, effectiveness in treating inflammatory conditions.

In addition, the disclosure provides methods of synthesizing DKPs. In one embodiment, the method comprises heating a mammalian plasma under conditions effective to cause the formation of a DKP. In an embodiment, the method comprises contacting plasma with an enzyme that cleaves the two N-terminal amino acids of the protein or peptide under conditions effective to produce a DKP. In an embodiment, the method comprises contacting plasma with DPP-IV that cleaves the two N-terminal amino acids of the protein or peptide under conditions effective to produce DA-DKP.

The disclosure further provides a method of making an improved pharmaceutical composition of a protein or peptide. The method comprises treating plasma so as to increase the content of DKPs such as DA-DKP in the pharmaceutical composition of a protein or peptide.

The disclosure also provides an improved pharmaceutical composition of a protein or peptide. The improvement is that the composition comprises an increased content of DKPs.

The preceding is a simplified summary to provide an initial understanding of the aspects, embodiments and configurations disclosed herein. This summary is neither an extensive nor exhaustive overview of the aspects, embodiments, or configurations. It is intended neither to identify key or critical elements, nor to delineate the scope of the aspects, embodiments, or configurations but to present selected concepts in a simplified form as an introduction to the more detailed description presented below. As will be appreciated, other aspects, embodiments, and configurations are possible utilizing, alone or in combination, one or more of the features set forth above or described in detail below.

### BRIEF DESCRIPTION OF DRAWINGS

The accompanying drawings are incorporated into and form a part of the specification to illustrate examples of how the aspects, embodiments, or configurations can be made and used and are not to be construed as limiting the aspects, embodiments, or configurations to only the illustrated and described examples. Further features and advantages will become apparent from the following, more detailed, description of the various aspects, embodiments, or configurations.

FIG. 1 illustrates DPP-IV activity in 5% commercial HSA solutions. DPP-IV activity (N=3) is represented as the total amount of p-nitroaniline (pNA, μM) produced during a 24 hour incubation at 37° C. Use of a DPP-IV inhibitor (diprotin A) resulted in the complete suppression of DPP-IV activity (data not shown).

FIG. 2 illustrates the effect of temperature on DPP-IV activity in a solution of 5% commercial HSA (CSL Behring). DPP-IV activity (N=3) is represented as the total amount of p-nitroaniline (pNA, μM) produced during a 2 hour incubation at 37° C. (solid bar) and 60° C. (vertical lines).

FIG. 3 illustrates DPP-IV activity in HSA solutions produced by different manufacturing methods. DPP-IV activity (N=3) is represented as the total amount of p-nitroaniline (pNA, μM) produced during a 24 hour incubation at 37° C. DPP-IV activity was measured in a commercial HSA solution produced using Cohn fractionation (solid bar, cHSA) and in a recombinant HSA solution produced in rice (vertical lines, rHSA).

FIG. 4 illustrates DA-DKP production in 5% commercial HSA heated at 60° C. DA-DKP production (N=3) was measured at different time points in a 5% commercial HSA solution heated at 60° C. in the presence (▴) or absence (▪) of a DPP-IV inhibitor. The low molecular weight fraction (<5 kDa) of the 5% commercial HSA solution was isolated and analyzed by LCMS for DA-DKP content. An asterisk (*) represents statistical significance (p<0.05) versus neat 5% HSA.

FIG. 5 illustrates one embodiment of the present disclosure, comprising two processing steps and one reaction step, which produce two separate DKP-containing product streams and one albumin-containing product stream.

FIG. 6 illustrates one embodiment of the present disclosure, similar to FIG. 5, comprising an albumin-containing recycle stream.

FIG. 7 illustrates an embodiment of the present disclosure, similar to FIG. 5, comprising a dilution stream to assist with DKP recovery during the second processing step.

### REFERENCE NUMERALS

| # | component |
|---|---|
| 100 | processing step |
| 110 | reacting step |
| 120 | feed stream |
| 130 | albumin-rich stream |
| 140 | albumin-lean stream |
| 150 | enzyme or catalyst |
| 160 | final albumin-rich product stream |
| 170 | albumin-rich recycle stream |
| 180 | diluent stream |

### DETAILED DESCRIPTION OF EMBODIMENTS

The following detailed description illustrates the invention by way of example and not by way of limitation. This description will clearly enable one skilled in the art to make and use the invention.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Human serum albumin (HSA) is the most abundant circulating protein with ligand binding and transport properties, antioxidant functions, and enzymatic activities. Because HSA is important for the regulation of blood volume and osmotic pressure in the critically ill, it is produced in mass quantities by the pharmaceutical industry. The preferred manufacturing technique of commercial HSA is based on the method of Cohn and colleagues which isolates HSA using a cold ethanol fractionation process. Commercial preparations of HSA usually contain the stabilizers N-acetyl-tryptophan (NAT) and sodium caprylate at concentrations of 0.08 mmol/g of HSA. The shelf life for commercial solutions of HSA is commonly 3 years. Due most likely to the production of reactive oxygen species, some age-related changes in the solution properties have been observed such as color changes, protein oxidation, proteolysis, aggregation, and precipitation. As a result, the stabilizer NAT is oxidized over time resulting in the production of two major degradation products with no known toxicity data available.

Since the Cohn fractionation process is not specific for HSA, some proteins and peptides are co-purified with HSA and are therefore present in commercial solutions. Additionally, since HSA has the unique ability to bind multiple ligands, other peptides and proteins with known biological activity have been identified in commercial solutions of HSA using proteomic techniques. These co-purified or bound proteins include proteases (kallikrein, cathepsin, carboxypeptidases, and dipeptidases), protease inhibitors (kininogen), cell surface adhesion proteins (selectin, cadherins, and ICAMs), and proteins involved in immunity (immunoglobulin chains and components of the complement system). Recently, a unique intrinsic proteolytic activity of the HSA molecule under reducing conditions has been documented. Therefore, due to its heterogeneous nature, the administration of HSA could introduce potentially unwarranted side effects to the critically ill patient.

In addition to proteins, commercial solutions of HSA contain a small immunosuppressive molecule derived from the first two N-terminal amino acids of HSA, aspartate-alanine diketopiperazine or DA-DKP. DA-DKP is thought to modulate T-cell cytokine production by increasing Rap1 activity and decrease activation factors relevant to the T-cell receptor signal transduction pathway. The mechanism of formation of DA-DKP in commercial solutions of HSA is currently unknown with one theory suggesting the auto-degradation of the N-terminus of HSA and subsequent formation of DA-DKP due to the unique chemical characteristics of the N-terminus.

The present disclosure is based on the existence of proteases in commercial solutions of HSA, specifically dipeptidyl peptidase IV (DPP-IV). As demonstrated in the examples below, using a known chromogenic assay, DPP-IV activity was measured in three commercial solutions of HSA. Also, this activity was abolished by the use of a known inhibitor of DPP-IV, diprotin A. Therefore, in addition to the presence of the DPP-IV protein, DPP-IV activity is also present in commercial solutions of HSA. This activity was not present in a recombinant HSA suggesting that the observed DPP-IV activity was a result of the Cohn fractionation process.

During the production of commercial HSA, the product is pasteurized for 10-11 hours by heating at 60° C. Optimum DPP-IV activity has been reported between 50 and 60° C. in serum, recombinant, and seminal DPP-IV with a gradual loss in activity at 65° C. This unique characteristic of DPP-IV makes it a candidate for the production of DA-DKP in commercial HSA solutions. Also, the low molecular weight components (other than bound to HSA) are most likely removed prior to the pasteurization step. Therefore, the majority of DA-DKP measured in commercial solutions of HSA is produced de novo from the pasteurization step onwards. In the commercial HSA solutions studied, significant DPP-IV activity was measured at 60° C. However, the total activity was only 70-80% of the activity present in the 37° incubations.

The production of DA-DKP in commercial HSA at 60° C. was examined using an LCMS method for detecting DA-DKP. In the neat solutions of commercial HSA, DA-DKP was produced in significant quantities over 24 hours at 60° C. When the DPP-IV inhibitor diprotin A was added to the commercial HSA solutions, the amount of DA-DKP produced at 60° C. decreased ˜3 fold over the 24 hour period. Therefore, this finding indicates that DPP-IV is partially responsible for the formation of DA-DKP in commercial solutions of HSA. Diprotin A did not completely abolish DA-DKP formation at 60° C. Diprotin A is trapped as a tetrahedral intermediate covalently bound to Ser630 inside the active site of DPP-IV. Diprotin A (Ile-Pro-Ile) is a substrate of DPP-IV with a low turnover leading to an apparent competitive inhibition. It is possible that diprotin A is hydrolyzed to a sufficient degree after a 24 hour incubation at 60° C. to allow other DPP-IV substrates into the active site such as the N-terminus of HSA. In combination with the enzymatic formation of DA-DKP, it is possible that of DA-DKP is formed via the auto-degradation of the N-terminus of HSA.

The known substrates of DPP-IV include several chemokines, cytokines, neuropeptides, circulating hormones and bioactive peptides. One of the most studied DPP-IV substrates is glucagon-like peptide 1 (GLP-1) which regulates circulating plasma glucose levels and is therefore important in the etiology of type II diabetes. Previously known DPP-IV substrates are polypeptides, and the N-terminus of HSA was first described as a substrate by the present inventor. Access of the N-terminus of HSA to the DPP-IV active site is unlikely to occur with HSA in its native confirmation due to steric hindrance. However, a significant portion of the HSA N-terminus needs to be accessible to the DPP-IV active site in order to form DA-DKP.

While not wishing to be bound by any theory, there are at least two ways in which the N-terminus of HSA can be presented to the active site of DPP-IV. First, the oxidation of HSA in commercial solutions during storage could cause the cleavage of HSA resulting in the production of N-terminal peptides that are better substrates for the DPP-IV active site. Redox active metals such as iron and copper are found in significant quantities in solutions of commercial HSA. Indeed, the N-terminus of HSA binds copper which can result in the in situ production of reactive oxygen species (ROS) possibly leading to the cleavage of HSA N-terminal peptides. Second, slow denaturation of HSA can result in the unfolding of the N-terminus making it a more accessible DPP-IV substrate. This is partially supported by the fact that at 60° C. HSA is in a reversible unfolded form possibly exposing the N-terminus. This reversible unfolded form may become more common during the prolonged storage of solutions of HSA leading to the increased production of DA-DKP.

The immunosuppressive capabilities of administrated HSA are well documented. In a rat model of hemorrhagic shock, HSA reduced lung permeability and neutrophil sequestration in a dose-dependent fashion. In a similar rat model of shock, administered HSA significantly down-regulated the expressions of integrins and ICAM-1, factors involved in the adhesion of immune cells to the endothelium. HSA also suppressed the respiratory burst of neutrophils in response to TNFα or complement exposure resulting in the selective and reversible inhibition of neutrophil spreading. Finally, HSA was found to be the least pro-inflammatory of the resuscitation fluids utilized in a hemorrhagic shock model. Based on previous immunological studies by the present inventor, DA-DKP appears to be partially responsible for the immunosuppressive capabilities of HSA.

The heterogeneity of commercial solutions of HSA can cause many beneficial or detrimental effects in a critically ill patient dependent on the immunological state of the patient. Some of the compounds recently identified in commercial solutions of HSA are involved in immune regulation and function. Additionally, the stabilizer NAT is a well-known antagonist of the neurokinin-1 receptor, an important mediator of the immune and inflammatory response as well as vascular permeability. The present disclosure deals with the mechanism of formation of the anti-inflammatory DA-DKP which is found in micromolar concentrations in commercial solutions of HSA. Commercial solutions of HSA contain significant levels of DPP-IV activity which is inhibited by diprotin A, a known DPP-IV inhibitor. Also, DPP-IV activity is unique to the commercial HSA solutions due to the Cohn manufacturing process which isolates other plasma components such as DPP-IV. Finally, the de novo formation of DA-DKP in heated commercial HSA solutions is observed with a corresponding inhibition of formation in the presence of diprotin A. Therefore, in commercial solutions of HSA, the peptidase DPP-IV appears to be involved in the formation of DA-DKP, a known anti-inflammatory compound.

Another aspect of the present disclosure involves a method for treating a feed stream comprising albumin and DKP, such as DA-DKP to produce compositions, the method comprising processing the feed stream to produce a first albumin-lean stream and a first albumin-rich stream, wherein the first albumin-lean stream comprises a first portion of the DKP present in the feed stream, and the first albumin-rich stream comprises a second portion of the DKP present in the feed stream. The first albumin-rich stream is reacted in order to produce additional DKP, resulting in a reaction stream comprising albumin and DKP. The reaction stream is processed to produce a second albumin-lean stream and a second albumin-rich stream, wherein the second albumin-lean stream comprises a portion of the DKP present in the reaction stream, and the second albumin-rich stream comprises a second portion of the DKP present in the reaction stream.

In some embodiments of the present disclosure, the albumin-rich streams produced can have therapeutic value in treating conditions that are conventionally treated by commercial HSA preparations, including but not limited to, effectiveness in treating hypoalbuminaemia and hypovolemia. In some embodiments of the present disclosure, the albumin-lean, DKP-containing streams produced can have therapeutic value, including but not limited to, effectiveness in treating inflammatory conditions.

In some embodiments of the present disclosure, at least two albumin-lean streams containing DKP are combined into a single stream.

Reference herein to a feed stream is to any aqueous solution that contains albumin and DKP. As such, the term “albumin” includes commercially available albumin preparations, such as albumin solutions produced by the Cohn process, variations thereof, chromatography, and any other suitable means to produce therapeutic proteins for human or animal use. The term “albumin” also refers to albumin from any species, including without limitation, human and bovine albumin. “Albumin” also includes albumin protein produced by synthetic methods such as by recombinant technology and/or cell expression systems using bacterial or mammalian expression hosts.

In some embodiments of the present disclosure, the concentration of albumin in an albumin- and DKP-containing feed stream can range from about 1 wt. % to about 35 wt. %. In some further embodiments, the concentration of albumin in the feed stream is in a range of from about 2 wt. % to about 30 wt. %. In still further embodiments, the concentration of albumin in the feed stream is in a range of from about 4 wt. % to about 26 wt. %. In particular embodiments, the concentration of albumin can be about 5 wt. % or about 25 wt. %.

Reference herein to a “diketopiperazine” or DKP, refers to any of the compounds having the following formula:

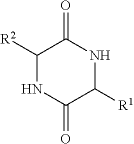

wherein R¹ and R² can be the same or different, and each is a side chain of an amino acid, wherein the amino acid is glycine, alanine, valine, norvaline, a-aminoisobutyric acid, 2,4-diaminobutyric acid, 2,3-diaminobutyric acid, leucine, isoleucine, norleucine, serine, homoserine, threonine, aspartic acid, asparagine, glutamic acid, glutamine, lysine, hydroxylysine, histidine, arginine, homoarginine, citrulline, phenylalanine, p-aminophenylalanine, tyrosine, tryptophan, thyroxine, cysteine, homocysteine, methionine, penicillamine or ornithine; provided, however, that when R¹ is the side chain of asparagine or glutamine, then R² cannot be the side chain of lysine or ornithine, and when R¹ is the side chain of lysine or ornithine, then R² cannot be the side chain of asparagine or glutamine.

In some embodiments of the present disclosure, the DKP (e.g., present in at least one of a feed stream, an albumin-containing stream, a DKP-containing stream, an albumin-rich stream, an albumin-lean-stream, and combinations thereof) can comprise at least one of aspartic acid-alanine diketopiperazine (DA-DKP), methionine-arginine diketopiperazine (MR-DKP), glutamic acid-alanine diketopiperazine (EA-DKP), tyrosine-glutamic acid diketopiperazine (YE-DKP), glycine-leucine diketopiperazine (GL-DKP), proline-phenylalanine diketopiperazine (PF-DKP) alanine-proline diketopiperazine (AP-DKP) and combinations thereof. In some embodiments of the present disclosure, the DKP (e.g., present in at least one of a feed stream, an albumin-containing stream, a DKP-containing stream, an albumin-rich stream, an albumin-lean-stream, and combinations thereof) can comprise DA-DKP, also known as 3-methyl-2,5-diketopiperazine-6-acetic acid, i.e., wherein R¹ is —CH₂—COOH and R² is —CH₃.

In some embodiments of the present disclosure, the feed stream can comprise DKP concentrations ranging from about 0 μM DKP to about 200 μM DKP. In still further embodiments of the present disclosure, the feed stream can comprise DKP concentrations ranging from about 50 μM DKP to about 100 μM DKP. In some embodiments of the present disclosure, the DKP is at least 50% DA-DKP, at least 60% DA-DKP, at least 70% DA-DKP, at least 80% DA-DKP, at least 90% DA-DKP, at least 95% DA-DKP, at least 98% DA-DKP, at least 99% DA-DKP, at least 99.9% DA-DKP or 100% DA-DKP.

In some embodiments of the present disclosure, processing at least one of the feed stream and the reaction stream can comprise protein separation techniques to separate protein, e.g. albumin, in an incoming stream into a protein rich stream. Such techniques can include at least one of filtration, chromatography, precipitation, extraction, and combinations thereof. In some embodiments of the present disclosure, processing at least one of the feed stream and the reaction stream can comprise filtration. In still further embodiments, processing at least one of the feed stream and the reaction stream can comprise tangential filtration.

Reference herein to filtration is to the mechanical and/or physical operation of separating one fraction of the albumin-containing feed stream from the remaining fraction by use of a pressure drop across a filtration media. The term “mechanical filtration” as used herein refers to, but is not limited to, size exclusion filtration. The term “physical filtration” as used herein refers to, but is not limited to, molecular interactions such as charge attraction and repulsion forces, hydrogen bonding, and dipole interactions. Filtration media can include, but is not limited to, filter paper, glass fibers, sintered glass, sintered metals, monolithic ceramics, polymeric membranes, and any one of these with or without a filter aid such as, but not limited to, diatomaceous earth. Filtration media can be hydrophilic and/or hydrophobic.

In some embodiments of the present disclosure, filtration can comprise tangential flow filtration. As used herein, the term “tangential flow” refers to the direction of flow of the albumin-containing feed stream relative to the filtration media. This flow direction can be either tangential (also commonly referred to as “cross flow”), or “normal flow”, or a combination of both. Tangential flow refers to an albumin-containing feed stream characterized by most of the stream flowing across the filtration media surface, whereas normal flow refers to a stream characterized by most of the stream flowing thru the filtration media, at a 90° angle relative to the filtration media surface.

In some embodiments of the present disclosure, a pressure drop for either type of filtration, or to cause flow through other processing unit operations (e.g., chromatography), can be accomplished by pressurizing at least one of the feed stream and the reaction stream using a pump, or by subjecting the down-stream-side of the filtration media to vacuum, or by subjecting the filter media and the at least one of the feed stream and the reaction stream to centrifugal forces, or by any other suitable means, or combinations thereof. As used herein, “down-stream-side” refers to the side of the filter media comprising the DKP-containing stream, or filtrate (also referred to as “albumin lean” and “DKP-containing side”), versus the “up-stream-side” or albumin-containing stream, which refers to the side of the filter media comprising the retentate (also referred to as “albumin rich” and “albumin-containing side”). As used herein, “vacuum” refers to an absolute pressure of less than 14.7 pounds per square inch absolute (psia).

In some embodiments of the present disclosure, processing can comprise chromatography. Reference herein to “chromatography” is to the mechanical and/or physical operation of separating one fraction of at least one of the feed stream and the reaction stream from the remaining fraction by use of a pressure drop across a stationary phase. The term “mechanical chromatography” as used herein refers to, but is not limited to, size exclusion chromatography. The term “physical chromatography” as used herein refers to, but is not limited to, affinity chromatography, ion exchange chromatography, fast protein liquid chromatography and immunoaffinity chromatography.

The stationary phase of a chromatography step, can include, but is not limited to, resins (i.e., polystyrene, polystyrene divinylbenzene and polyacrylamide), ion exchange resins (i.e., sulfonated, quaternary ammonium, carboxylate and diethyl ammonium functional groups), cross-linked agarose, cross-linked dextrans, phosphocellulose, porous glass and silica, alumina and zirconia matrices. Further, the stationary phase can be immobilized on a solid support particle, or on the inner wall of a cylinder, either by physical attraction, chemical bonding, and or by in situ polymerization after coating. The immobilized stationary phase can coat the outer surfaces of the particles and cylinder, and/or fill any available pores within the solid particles. The bonded stationary phase can be selected from the group consisting of, but not limited to, polymeric-bonded, polymer-grafted, capped stationary, alkyl-bonded, phenyl-bonded, cyano-bonded, diol-bonded, and amino-bonded stationary phases, all of which are terms known to one of ordinary skill in the art of chromatography. Further, the stationary phase can be functionalized with biospecific ligands which include, but are not limited to, antibodies, protein receptors, steroid hormones, vitamins and enzyme inhibitors.

In some embodiments of the present disclosure, the stationary phase can be housed and immobilized in a chromatography column. The at least one feed stream and reaction stream can be fed to the inlet of the chromatography column, with albumin-rich and albumin-lean streams exiting at the outlet of the column, wherein separation of the albumin-rich and albumin-lean streams can be accomplished by differing elution times. Pressure drop for delivering the feed stream through the chromatography column can be accomplished by pressurizing the at least one feed stream and reaction stream using at least one pump.

In some embodiments of the present disclosure, processing can comprise a size exclusion process wherein a feed stream or reaction stream or both is separated into an albumin-rich retentate stream and an albumin-lean filtrate stream containing DKP. In some embodiments of the present disclosure, the retentate retains greater than about 80 wt %, greater than about 85 wt %, greater than about 90 wt %, greater than about 95 wt %, or greater than about 99 wt %, of the proteins present in the albumin- and DKP-containing feed stream, including proteins with a molecular weight greater than about 10 kDa, 20 kDa, 30 kDa, 40 kDa, 50 kDa, 60 kDa, 70 kDa, 80 kDa, 90 kDa or 100 kDa.

In some embodiments of the present disclosure, reacting the DKP can comprise at least one of thermal, chemical, enzymatic processing, and combinations thereof.

In some embodiments of the present disclosure, reacting an albumin-containing stream can comprise at least one of heat-treating, pasteurizing, enzymatically reacting, chemically reacting, and combinations thereof. In some embodiments of the present disclosure, reacting an albumin-containing stream can comprise heating the albumin-containing stream to an average bulk temperature ranging from about 40° C. to about 80° C. In some embodiments of the present disclosure, reacting an albumin-containing stream can comprise heating the albumin-containing stream to an average bulk temperature ranging from about 50° C. to about 70° C. In some embodiments of the present disclosure, reacting an albumin-containing stream can comprise heating the albumin-containing stream to an average bulk temperature ranging from about 55° C. to about 65° C. In some embodiments of the present disclosure, reacting an albumin-containing stream can comprise heating the albumin-containing stream to an average bulk temperature ranging from about 57.5° C. to about 62.5° C. In some embodiments of the present disclosure, reacting an albumin-containing stream can comprise heating the albumin-containing stream to an average bulk temperature of about 60° C.

In some embodiments of the present disclosure, reacting an albumin-containing stream can comprise enzymatically reacting the albumin-containing stream with at least one dipeptidase, kallikrein, cathepsin, carboxypeptidase, and combinations thereof. In some further embodiments of the present disclosure, reacting an albumin-containing stream can comprise enzymatically reacting the stream with at least dipeptidyl peptidase IV.

In some embodiments of the present disclosure, the at least one dipeptidase, kallikrein, cathepsin, carboxypeptidase, and combinations thereof, can be present in the feed stream as received from a commercial albumin supplier, or a non-commercial albumin supplier. For example, an albumin-containing feedstock can contain enzymatically active dipeptidases which are capable of producing further DKP in a subsequent reaction step, or over the course of time while, for example, kept in storage at ambient conditions. In some embodiments of the present disclosure, a feed stream can comprise dipeptidase wherein the dipeptidase activity, as measured in an assay using the chromogenic substrate, Gly-Pro-pNA as described in the examples, ranges from more than 0 μM pNA to about 200 μM pNA. In some further embodiments of the present disclosure, a feed stream can comprise dipeptidase wherein the dipeptidase activity ranges from about 40 μM pNA to about 140 μM pNA.

In some further embodiments of the present disclosure, the at least one dipeptidase, kallikrein, cathepsin, carboxypeptidase, and combinations thereof, can be added to at least one of a feed stream, a first albumin-rich stream, a second albumin-rich stream, any subsequent albumin-rich streams, and combinations thereof. In some embodiments of the present disclosure, a dipeptidase can be added to at least one of a feed stream, a first albumin-rich stream, a second albumin-rich stream, any subsequent albumin-rich streams, and combinations thereof. In still further embodiments of the present disclosure, a dipeptidase can be added to at least one of a feed stream, a first albumin-rich stream, a second albumin-rich stream, any subsequent albumin-rich streams, and combinations thereof, wherein the peptidase activity can be increased to be from about 0 μM pNA to about 200 μM pNA. In still further embodiments, the peptidase activity can be increased to be from about 40 μM pNA to about 150 μM pNA

In some further embodiments of the present disclosure, reacting an albumin-rich stream can comprise the catalytic reaction of albumin present in an albumin-rich stream with at least one redox-active metal, such as iron and copper. Other potential metal catalysts include, but are not limited to, lithium, potassium, calcium, sodium, magnesium, aluminum, zinc, nickel, lead, manganese, tin, silver, platinum, gold, and combinations thereof. In some embodiments of the present disclosure, reacting an albumin-rich stream can comprise at least one redox-active metal present as a homogeneous catalyst, a heterogeneous catalyst, or both. In some further embodiments of the present disclosure, the reacting an albumin-rich stream can comprise passing the stream through packed-bed reactor comprising a solid catalyst comprising at least one redox-active metal supported on a substrate. In some further embodiments of the present disclosure, the reacting an albumin-rich stream can comprise reacting the albumin in a slurry reactor, wherein the redox-active metal is suspended in a liquid mixture and/or mixed using a means for mixing.

In some embodiments of the present disclosure, a reactor for reacting an albumin-rich stream can comprise a batch reactor, a continuous reactor, and combinations thereof. In some further embodiments, a reactor can comprise a stirred-tank reactor, a continuous stirred-tank reactor, a packed-bed reactor, a plug-flow reactor, and combinations thereof.

In some embodiments of the present disclosure, reacting an albumin-rich stream can comprise heating an albumin-rich stream to a bulk temperature higher than ambient temperature. For example only, in some embodiments, reacting an albumin-rich stream can comprise heating the stream to temperatures less than temperatures where albumin and DKP are denatured and greater than about 20° C., greater than about 30° C., greater than about 40° C., greater than about 50° C., greater than about 60° C., greater than about 70° C., or greater than about 80° C. In some still further embodiments of the present disclosure, reacting an albumin-rich stream can comprise both heating the albumin-rich stream and at least enzymatically reacting and/or chemically reacting the albumin-rich stream.

In some embodiments of the present disclosure, in addition to albumin and DKP, a feed stream can include a number of additional components. Such components can be naturally occurring species derived from the blood from which the albumin solution is produced, or they can be species occurring from a method of synthesis of synthetically produced albumin, or they can be species introduced or produced during purification of a natural product, for example, but not limited to, purification of blood plasma using the Cohn process and variations thereof. Species introduced to the albumin-containing feed stream can include additives intentionally added to the albumin-containing feed streams, either pre- or post-synthesis of synthetic albumin, or pre- or post-purification of naturally occurring albumin. Such additives include, but are not limited to sodium, potassium, N-acetyltryptophan, sodium caprylate and/or caprylic acid. Species produced during purification of a natural albumin product include, but are not limited to, amino acids, DKPs and any other compound or species resulting from thermal, physical, enzymatic or chemical degradation of naturally occurring plasma proteins.

In some embodiments of the present disclosure, the first albumin-rich stream can comprise at least about 10%, at least about 20%, at least about 30%, at least about 40%, at least about 50%, at least about 60%, at least about 70%, at least about 80%, at least about 90%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.91%, at least about 99.92%, at least about 99.93%, at least about 99.94%, at least about 99.95%, at least about 99.96%, at least about 99.97%, at least about 99.98%, at least about 99.99%, by weight of the albumin in the feed stream.

By way of example, for a case wherein processing an albumin-containing feed stream results in an albumin-containing stream comprising at least about 90% by weight of the albumin in the feed stream, if the albumin-containing feed stream comprises 100 milliliters of albumin-containing feed, at an albumin concentration of 0.03 grams albumin per milliliter, the product stream resulting from the processing step (i.e., filtration, chromatography, etc.) comprises at least 2.7 grams of albumin. Similarly by way of example, if the albumin-containing feed stream comprises 100 milliliters of albumin-containing feed, at an albumin concentration of 0.5 grams albumin per milliliter, the resultant albumin-containing stream comprises at least 45 grams of albumin. It would be obvious to one of ordinary skill in the art, that scaling the above exemplary volumes and/or percentages up or down, will result in corresponding changes to the amounts of albumin present in the albumin-rich and albumin-lean streams, as calculated using simple mathematics.

In some embodiments of the present disclosure, the second albumin-rich stream can comprise at least about 10%, at least about 20%, at least about 30%, at least about 40%, at least about 50%, at least about 60%, at least about 70%, at least about 80%, at least about 90%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.91%, at least about 99.92%, at least about 99.93%, at least about 99.94%, at least about 99.95%, at least about 99.96%, at least about 99.97%, at least about 99.98%, at least about 99.99%, by weight of the albumin in the reaction stream.

In some embodiments of the present disclosure, a subsequent albumin-rich stream, produced by a processing step other than the first two processing steps, can comprise at least about 10%, at least about 20%, at least about 30%, at least about 40%, at least about 50%, at least about 60%, at least about 70%, at least about 80%, at least about 90%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.91%, at least about 99.92%, at least about 99.93%, at least about 99.94%, at least about 99.95%, at least about 99.96%, at least about 99.97%, at least about 99.98%, at least about 99.99%, by weight of the albumin present in the albumin-rich stream that feeds the processing step other than the first two processing steps.

In some embodiments of the present disclosure, the first portion of DKP present in the first albumin-lean stream can comprise at least about 5% by weight, at least about 10% by weight, at least about 20% by weight, at least about 30% by weight, at least about 40% by weight, at least about 50% by weight, at least about 60% by weight, at least about 70% by weight, at least about 80% by weight, at least about 90% by weight, or at least about 99% by weight, of the DKP present in the feed stream.

In some embodiments of the present disclosure, the first portion of DKP present in the second albumin-lean stream can comprise at least about 5% by weight, at least about 10% by weight, at least about 20% by weight, at least about 30% by weight, at least about 40% by weight, at least about 50% by weight, at least about 60% by weight, at least about 70% by weight, at least about 80% by weight, at least about 90% by weight, or at least about 99% by weight, of the DKP present in the reaction stream.

In some embodiments of the present disclosure, a subsequent portion of DKP present in a subsequent albumin-lean stream, due to a processing step other than the first two processing steps, can comprise at least about 5% by weight, at least about 10% by weight, at least about 20% by weight, at least about 30% by weight, at least about 40% by weight, at least about 50% by weight, at least about 60% by weight, at least about 70% by weight, at least about 80% by weight, at least about 90% by weight, or at least about 99% by weight, of the DKP present in the albumin-rich stream that feeds the processing step other than the first two processing steps.

In some embodiments of the present disclosure the first albumin-lean stream can comprise DKP concentrations of at least about 10 μM, at least about 20 μM, at least about 30 μM, at least about 40 μM, at least about 50 μM, at least about 60 μM, at least about 70 μM, at least about 80 μM, at least about 90 μM, at least about 100 μM, at least about 110 μM, at least about 120 μM, at least about 130 μM, at least about 140 μM, at least about 150 μM, at least about 160 μM, at least about 170 μM, at least about 180 μM, at least about 190 μM, at least about 200 μM, at least about 250 μM, at least about 300 μM, at least about 350 μM, at least about 400 μM, at least about 450 μM, or at least about 500 μM. In still further embodiments, the second albumin-lean stream can comprise DKP concentrations of at least about 10 μM, at least about 20 μM, at least about 30 μM, at least about 40 μM, at least about 50 μM, at least about 60 μM, at least about 70 μM, at least about 80 μM, at least about 90 μM, at least about 100 μM, at least about 110 μM, at least about 120 μM, at least about 130 μM, at least about 140 μM, at least about 150 μM, at least about 160 μM, at least about 170 μM, at least about 180 μM, at least about 190 μM, at least about 200 μM, at least about 250 μM, at least about 300 μM, at least about 350 μM, at least about 400 μM, at least about 450 μM, or at least about 500 μM.

A further aspect of the present disclosure, is a method for treating a feed stream comprising albumin and DKP to produce therapeutic compositions, as described above, further comprising an analyzing step, wherein the analyzing step comprises analyzing an albumin-rich stream to yield at least one metric, comparing the at least one metric to at least one reference value, wherein when the at least one metric is greater or less than the reference value, the reacting and processing steps are repeated until the at least one metric of a subsequent albumin-rich stream is equal to or less or greater than the at least one reference value.

In some embodiments of the present disclosure, the analyzing step can comprise high pressure liquid chromatography and mass-spectroscopy, or any other suitable analytical method for measuring a metric of interest. In some embodiments of the present disclosure, the at least one metric is the mass of full length albumin remaining after at least one processing step, and the reference value is a fraction of a theoretical maximum mass of albumin that can be processed to produce DA-DKP. In other embodiments, the at least one metric is the mass of DKP produced in at least one processing step, and the reference value is a fraction of a theoretical maximum mass of DKP that can be produced from the albumin in the feed stream.

In some embodiments of the present disclosure, a method for treating a feed stream comprising albumin and DKP to produce therapeutic compositions can further comprise adjusting the pH of an albumin-rich stream. In some embodiments of the present disclosure, a feed stream can be pH adjusted. In some further embodiments, an albumin-rich stream is pH adjusted prior to a reacting step and/or during a reacting step. In some further embodiments, an albumin-rich stream is pH adjusted prior to a processing step and/or during a processing step. The pH of an albumin-rich stream can be adjusted to improve at least one of an enzymatic reaction, a catalytic reaction, heat degradation, and combinations thereof. In some embodiments of the present disclosure, adjusting the pH of an albumin-rich stream can comprise adjusting the pH to a range from about 1.5 to about 10.0. In some further embodiments of the present disclosure, adjusting the pH of an albumin-rich stream can comprise adjusting the pH to a range from about 4.0 to about 8.0. In still further embodiments of the present disclosure, the pH of an albumin-rich stream is adjusted to about physiological pH, i.e., to about pH 7.3-7.4.

In some embodiments of the present disclosure, a method for treating a feed stream comprising albumin and DKP to produce therapeutic compositions can further comprise diluting an albumin-rich stream. In some further embodiments of the present disclosure, at least one of the feed stream and the reaction stream can be diluted. In still further embodiments of the present disclosure, diluting can be achieved using a diluent selected from the group consisting of saline, Lactated Ringer's solution, Ringer's acetate solution, hydroxyethyl starch solutions and dextrose solutions.

A dilution step can provide a means for adding additional components, either to albumin-lean streams and/or to albumin-rich streams, which possess a variety of additional therapeutic values. For example, Lactated Ringer's solution can be added as a diluent to an albumin-lean stream, rich in DKP, to assist with controlling metabolic acidosis in an immune-compromised patient. Other solutions can be selected as diluents, either individually or as mixtures, to meet the specific therapeutic requirements of a particular patient or demographic, an added to either or both of an albumin-rich stream and an albumin-lean stream.

In addition, a dilution step can provide a diluent that provides a displacement volume to enable higher recovery percentages of the DKP present in the starting albumin feed material. In some embodiments of the present disclosure, the processing step can comprise a size exclusion separation, wherein essentially all of the albumin present in a feed stream is retained in a retentate. Conversely, in these embodiments, essentially none of the albumin present in the feed stream passes through the size exclusion separation unit with the filtrate. In this scenario, the albumin can be viewed as the particulate in a slurry, with the remaining DKP-containing aqueous phase as the liquid suspending the albumin particulate in the slurry. Thus, the filtrate is essentially the same DKP-containing aqueous phase as what remains in the retentate. Furthermore, in this scenario a single-stage, or even multiple-staged size exclusion unit operations, is not able to completely remove all of the DKP-containing aqueous phase from the albumin. Without some assistance, the albumin-rich stream will retain some of the DKP-containing aqueous phase. A diluent can provide a liquid volume that can flush and displace a percentage of this DKP-containing aqueous phase from the albumin, through the size exclusion unit operation and into the retentate, or albumin-lean stream.

A further aspect of the present disclosure is a composition comprising DKP that contains less than 10 weight percent albumin. In some embodiments of the present disclosure, the concentration of albumin in the DKP-containing composition can be less than about 1 weight percent or less than about 0.1 weight percent. In still further embodiments of the present disclosure, the concentration of albumin in the DKP-containing composition can be about zero weight percent, or at non-detectable limits.

In some embodiments of the present disclosure, the composition comprising DKP may provide therapeutic benefits such as, but not limited to, effectiveness in treating inflammatory conditions.

In some embodiments of the present disclosure, the DKP can comprise at least one of aspartic acid-alanine DKP, methionine-arginine DKP, glutamic acid-alanine DKP, tyrosine-glutamic acid DKP, glycine-leucine DKP, proline-phenylalanine DKP, alanine-proline DKP, and combinations thereof. In still further embodiments, the DKP can be present in a concentration ranging from about 25 μM DKP to about 200 μM DKP.

In some embodiments of the present disclosure, the composition comprising DKP can further comprise at least one of saline, Lactated Ringer's solution, Ringer's acetate solution, hydroxyethyl starch solution, dextrose solutions, and combinations thereof. In some embodiments of the present disclosure, the composition comprising DKP can further comprise at least one additional component selected from the group consisting of sodium acetyltryptophanate, N-acetyltryptophan, caprylic acid and salts thereof such as sodium caprylate, and combinations thereof. Such additional components can be present in amounts typically found in commercial HSA. For example, such components can be present in amounts from about 0.1 mM to about 30 mM or in ranges having a lower end of the range selected from about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, or about 20 mM. Such ranges can have a higher end of the range selected from about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, or about 35 mM.

The methods of the present disclosure advantageously provide increased amounts of DKPs more efficiently in comparison to methods of synthesizing DKPs that were previously known. In particular, these embodiments of methods of the present disclosure synthesize DKPs from mammalian plasma. The plasma may be from a mammal, such as a rabbit, goat, dog, cat, horse or human. The animal is preferably a human, and the plasma is preferably human plasma.

Plasma contains components for the synthesis of DKPs, including albumin, immunoglobulin, and erythropoietin, as well as other proteins and peptides. Methods of the present disclosure include synthesizing DKPs from plasma, where the methods of the present disclosure can advantageously increase the amount of DKPs synthesized when compared to prior art methods of producing DKPs.

HSA is a principal protein component present in plasma, consisting of a single chain polypeptide comprising 585 amino acid residues and has a molecular weight equal to about 66,000 Dalton (see Minghetti, P. P. et al. (1986), Molecular structure of the human albumin gene is revealed by nucleotide sequence within 11-22 of chromosome 4. J. Biol. Chem. 261, pp. 6747-6757). HSA has typically been prepared by subjecting the human plasma to Cohn fractionation, a low temperature ethanol fractionation method, or similar methods, to produce HSA-containing fractions (HSA is fractionated in the fraction V), and then purifying the fraction through the use of a variety of purification techniques. The HSA was then purified using one or more of a salting out method, an ultrafiltration method, and isoelectric precipitation method, an electrophoresis method, an ion-exchange chromatography technique, a gel filtration chromatography technique and/or an affinity chromatography technique.

When plasma is processed to produce HSA or other solutions of proteins and/or peptides, the processing reduces the amounts of albumin, immunoglobulin, and erythropoietin, and other proteins and peptides, which are available to form DKP. In other words, plasma has increased amounts of albumin, immunoglobulin, and erythropoietin, as well as other proteins and peptides, in comparison to HSA or other purified solutions of peptides or proteins. Thus, some of the methods of the present disclosure described herein use plasma to produce DKPs.

Accordingly, DKPs for use in the present disclosure can be prepared by heating plasma. “Plasma” may refer to unprocessed plasma, or a plasma solution in phosphate buffer at neutral pH. Preferably, the plasma solution is a concentrated solution (e.g., about 100-500 mM) to achieve protonation of the N-terminal and/or C-terminal amino acid. The plasma can be heated at 60° C. for at least about 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17, hours, 18 hours, 19 hours, 20 hours, 21 hours, 22 hours, 23 hours, 24 hours, 2 days, 3 days, 4 days, 5 days, 6 days, 7 days, 8 days, 9 days, 10 days, to cause formation of the DKPs. Denaturation of the protein should, preferably, be avoided. This can be accomplished by using shorter times and/or by adding caprylic acid or N-acetyl tryptophan at about 0.02 M for each.

DKPs for use in the present disclosure can also be prepared by contacting plasma with an enzyme that can cleave the two N-terminal amino acids from proteins or peptides (e.g., dipeptidyl peptidases, and in particular DPP-IV) in the plasma, or an enzyme that can cleave the two C-terminal amino acids from the protein or peptide (e.g., carboxypeptidases). The reaction should be conducted at pH 6-8, preferably in a buffer, such as phosphate buffer, at a temperature high enough to speed the reaction but not so high that the protein is denatured.

In an embodiment, the desired DKP is DA-DKP, the enzyme is DPP-IV, the temperature is from about 40° C. to about 80° C., and preferably about 60° C., the reaction time is from about 5 hours to about 6 days. In an embodiment the DPP-IV is endogenous, and is already in the plasma, is added to the plasma during the process or a combination thereof. The process temperature can be at least about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, and about 80° C. The reaction time can be at least about 5, about 6, about 7, about 8, about 9, about 10, about 11, about 12, about 13, about 14, about 15, about 16, about 17, about 18, about 19, about 20, about 21, about 22, about 23 or more hours, about 1, about 1.1, about 1.2, about 1.3, about 1.4, about 1.5, about 1.6, about 1.7, about 1.8 about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 6, about 7, about 8, about 9 or about 10 days.

The DKPs made by methods of the present disclosure can be purified from solutions containing them, including from the commercially-available pharmaceutical compositions comprising albumin, immunoglobulin and erythropoietin, by well known methods, such as size-exclusion chromatography (e.g., Centricon filtration), affinity chromatography (e.g., using a column of beads having attached thereto an antibody or antibodies directed to the desired DKP(s) or an antibody or antibodies directed to the truncated protein or peptide), anion exchange or cation exchange. The purified DKPs can be used and incorporated into pharmaceutical compositions as described above.

The DKPs include all possible stereoisomers that can be obtained by varying the configuration of the individual chiral centers, axes or surfaces. In other words, the DKPs include all possible diastereomers, as well as all optical isomers (enantiomers).

The physiologically-acceptable salts of the DKPs of the disclosure may also be used in the practice of the disclosure. Physiologically-acceptable salts include conventional non-toxic salts, such as salts derived from inorganic acids (such as hydrochloric, hydrobromic, sulfuric, phosphoric, nitric, and the like), organic acids (such as acetic, propionic, succinic, glycolic, stearic, lactic, malic, tartaric, citric, glutamic, aspartic, benzoic, salicylic, oxalic, ascorbic acid, and the like) or bases (such as the hydroxide, carbonate or bicarbonate of a pharmaceutically-acceptable metal cation or organic cations derived from N,N-dibenzylethylenediamine, D-glucosamine, or ethylenediamine). The salts are prepared in a conventional manner, e.g., by neutralizing the free base form of the compound with an acid.

As noted above, a DKP of the disclosure, or a physiologically-acceptable salt thereof, can be used to treat a T-cell mediated disease or to inhibit activation of T-cells. To do so, a DKP, or a physiologically-acceptable salt thereof, is administered to an animal in need of such treatment. Preferably, the animal is a mammal, such as a rabbit, goat, dog, cat, horse or human. Effective dosage forms, modes of administration and dosage amounts for the compounds of the disclosure may be determined empirically, and making such determinations is within the skill of the art. It is understood by those skilled in the art that the dosage amount will vary with the particular compound employed, the disease or condition to be treated, the severity of the disease or condition, the route(s) of administration, the rate of excretion of the compound, the duration of the treatment, the identify of any other drugs being administered to the animal, the age, size and species of the animal, and like factors known in the medical and veterinary arts. In general, a suitable daily dose of a compound of the present disclosure will be that amount of the compound which is the lowest dose effective to produce a therapeutic effect. However, the daily dosage will be determined by an attending physician or veterinarian within the scope of sound medical judgment. If desired, the effective daily dose may be administered as two, three, four, five, six or more sub-doses, administered separately at appropriate intervals throughout the day. Administration of the compound should be continued until an acceptable response is achieved.

The compounds of the present disclosure (i.e., DKPs and physiologically-acceptable salts thereof) may be administered to an animal patient for therapy by any suitable route of administration, including orally, nasally, rectally, vaginally, parenterally (e.g., intravenously, intraspinally, intraperitoneally, subcutaneously, or intramuscularly), intracisternally, transdermally, intracranially, intracerebrally, and topically (including buccally and sublingually). The preferred routes of administration are orally and intravenously.

While it is possible for a compound of the present disclosure to be administered alone, it is preferable to administer the compound as a pharmaceutical formulation (composition). The pharmaceutical compositions of the disclosure comprise a compound or compounds of the disclosure as an active ingredient in admixture with one or more pharmaceutically-acceptable carriers and, optionally, with one or more other compounds, drugs or other materials. Each carrier must be “acceptable” in the sense of being compatible with the other ingredients of the formulation and not injurious to the animal. Pharmaceutically-acceptable carriers are well known in the art. Regardless of the route of administration selected, the compounds of the present disclosure are formulated into pharmaceutically-acceptable dosage forms by conventional methods known to those of skill in the art. See, e.g., Remington's Pharmaceutical Sciences.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### Example 1

A proteomic analysis was performed on commercial HSA solutions in order to understand the therapeutic effects, adverse reactions, and mechanisms involved in treatments using HSA solutions. In this study, a total of 1219 peptides corresponding to 141 proteins different from HSA were identified. More importantly, the peptidase DPP-IV was positively identified in the commercial HSA solution. Therefore, due to its ability to cleave peptides after an alanine residue, it is conceivable that DPP-IV is involved in the formation of DA-DKP in commercial HSA solutions. To test this hypothesis, commercially available solutions of HSA were assayed for DPP-IV activity using a chromogenic substrate and known DPP-IV inhibitor. The presence of DPP-IV activity was also tested in a recombinant HSA source not produced via the Cohn fractionation process. Finally, the effect of temperature on DPP-IV activity as well as DA-DKP production in commercial solutions of HSA was assessed.

Materials and Methods

Materials.

Three commercially available 250 mL 5% HSA (w/v) products (CSL Behring LLC, Kankakee, Ill., USA; Grifols Biologicals Inc., Los Angeles, Calif., USA; Octapharma USA Inc., Hoboken, N.J., USA) were used throughout the study. The N-terminal HSA peptide (DAHK) was manufactured by Diosynth Inc. (Netherlands). Recombinant HSA (ecoHSA™) was obtained from Genlantis Inc. (San Diego, Calif., USA) and was produced in the seeds of Asian Rice (Oryza sativa). Synthetic DA-DKP was produced by Syngene International Ltd. (India). All other reagents including the DPP-IV substrate and inhibitor were obtained from Sigma-Aldrich Co. LLC (St. Louis, Mo., USA).

DPP-IV Assay.

DPP-IV activity was assayed by using a chromogenic substrate, Gly-Pro-pNA, as described in E. Nemoto, S. Sugawara, H. Takada, et al., Increase of CD26/dipeptidyl peptidase IV expression on human gingival fibroblasts upon stimulation with cytokines and bacterial components. Infect Immun 67 (1999) 6225-33. All reactions were carried out in DPP-IV assay buffer (pH 7.6) consisting of 0.1M HEPES, 0.12M NaCl, 5 mM KCl, 8 mM glucose, and 10 mg/ml bovine serum albumin (BSA). 5% commercial HSA, recombinant HSA, or buffer blank (0.9% NaCl) were combined with 1 mM Gly-Pro-pNA (DPP-IV substrate) in assay buffer or assay buffer only (−CON). Incubations were performed at 37° C. or 60° C. for 2-24 hours. For DPP-IV inhibition studies, 1 mM diprotin A in assay buffer was pre-incubated with the HSA solutions for 15 minutes at 37° C. prior to DPP-IV substrate addition. All incubations were read at 405 nm (SpectraMax M2 spectrophotometer, Molecular Devices LLC, Sunnyvale, Calif., USA). Each reading at 405 nm was corrected by subtracting the A405 for the DPP-IV substrate-containing incubation from the corresponding A405 for the −CON incubation for each HSA solution tested.

Isolation of <5 kDa HSA Fraction.

For the analysis of DA-DKP formation, an aliquot was added to a microcentrifugal filter (Vivaspin 2, MWCO 5,000, Sartorius Stedim Biotech, Goettingen, Germany). Filters were centrifuged at 3,500 rpm for 30 minutes at room temperature. The <5 kDa fraction was collected and transferred to a separate, storage tube for LCMS analysis.

LCMS Assay.

Each <5 kDa fraction & DA-DKP synthetic standard (20-2000 ng/mL) were spiked with 0.01 mM L-Tryptophan-d5 (indole-d5) which was used as an internal standard. 50 μL was injected into a strong anion exchange column (Spherisorb, S5 SAX 250 mm×4.0 mm, Waters, Milford, Mass., USA) connected to high performance liquid chromatography (HPLC, Waters 2795 Separations Module, Milford, Mass., USA) coupled to a mass spectrometer (LCT-TOF, Micromass, UK). A ternary mobile phase consisting of dH₂O (Solvent A), methanol (Solvent B), and 200 mM ammonium formate (pH 5.4, Solvent C) was used at a flow rate of 0.5 mL/min using the gradient below (Table 1).

TABLE 1
HPLC gradient used in the separation of DA-DKP in
>5kDa HSA solutions.

| Time (min) | A (%) | B (%) | C (%) |
|---|---|---|---|
| 0 | 25 | 40 | 35 |
| 10 | 10 | 40 | 50 |
| 15 | 10 | 40 | 50 |
| 15.01 | 25 | 40 | 35 |
| 20 | 25 | 40 | 35 |

The output of the HPLC was split 1:20 (v/v) and injected into the mass spectrometer using negative electrospray ionization (−ESI) with a scan range of 80 to 1000 m/z, cone voltage of 30 eV, source temperature of 100° C., and gas temperature of 300° C. DA-DKP was measured by monitoring [M]=185, which corresponds to DA-DKP minus a single proton (—H+). The straight chain of DA-DKP, Asp-Ala, can also be analyzed with this method by monitoring [M]=203.

Statistical Methods.

The amount of pNA produced in μM was calculated based on the pNA molar extinction coefficient in HEPES buffer (see R. Lottenberg, C. M. Jackson, Solution composition dependent variation in extinction coefficients for p-nitroaniline. Biochim Biophys Acta 742 (1983) 558-64). Statistical analysis was performed using the software packages Excel (Microsoft) and Matlab R13 (MathWorks). Groups were compared using a two tailed students' T-test with a significance level at p<0.05. All data is reported as mean±SD.

Results

Dipeptidyl peptidase IV (DPP-IV) activity was assessed in commercial preparations of human serum albumin (HSA). The activity assay chosen is well documented in the literature and involves the cleavage of a known DPP-IV substrate, Gly-Pro-pNA. The resulting liberation of a chromogen, pNA, was measured spectrophotometrically at 405 nm. Three commercially available solutions of 5% HSA were chosen with no particular manufacturer preference. The only requirements were that the solutions were unexpired and were produced by different manufacturers using the Cohn fractionation process. For the incubation temperatures of the enzyme assay, 37° C. and 60° C. were chosen since the former represents physiological conditions and the latter represents the pasteurization temperature of commercial HSA solutions.

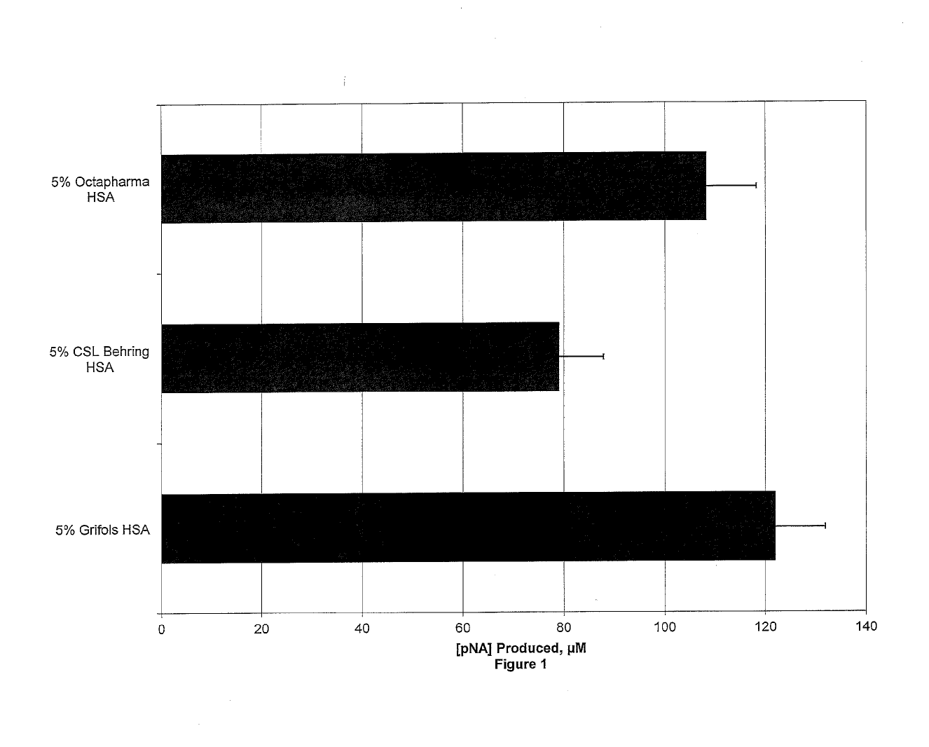

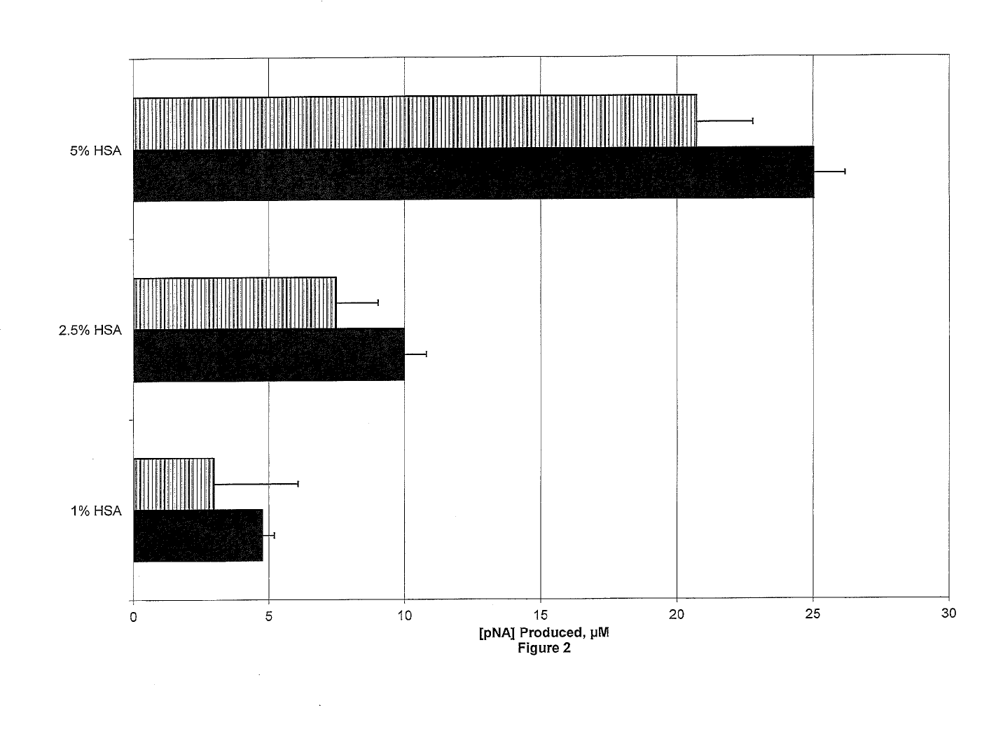

DPP-IV activity at 37° C. was measured in all three 5% commercial HSA solutions. All three commercial HSA solutions contained significant DPP-IV activity with the CSL Behring HSA having slightly less activity than the Octapharma and Grifols HSA (FIG. 1). The amount of DPP-IV activity did not correlate with the expiration dates of the HSA sources. DPP-IV was completely suppressed in the presence of a known DPP-IV inhibitor (diprotin A). This resulted in no additional chromogen production during the entire incubation compared to the −CON (data not shown). In one of the commercial HSA solutions (CSL Behring), DPP-IV activity at 60° C. was assayed. DPP-IV activity was present at significant levels (FIG. 2). However, DPP-IV activity at 60° C. was ˜70-80% of the original DPP-IV activity at 37° C. At both temperatures, a dose-response in DPP-IV activity was observed with increasing concentrations of the HSA solution.

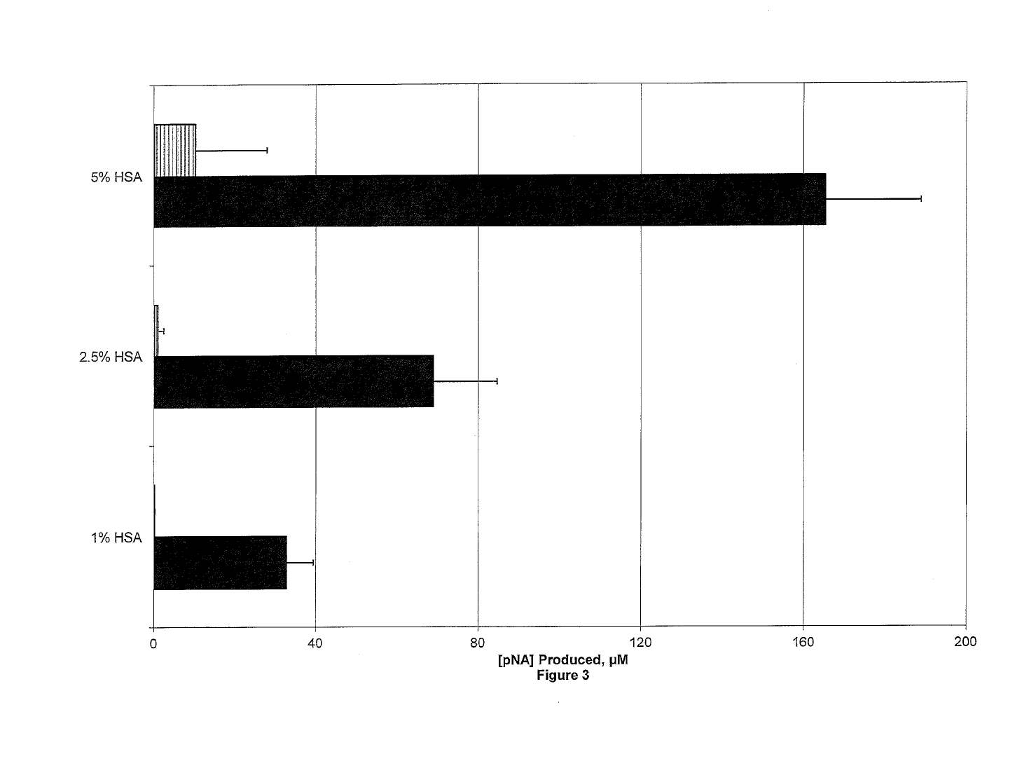

To compare DPP-IV activity in HSA isolated using a non-Cohn fractionation process, a recombinant HSA (rHSA) produced in rice was analyzed. One of the commercial HSA solutions produced by Cohn fractionation (cHSA) was also included in the DPP-IV activity assay. For both HSA types, concentrations ranged from neat (5% w/v) to diluted solutions (1% and 2.5%). At all three concentrations, the amount of DPP-IV activity in the cHSA solution was significantly higher than the rHSA solution (FIG. 3). Also, DPP-IV activity in the rHSA solutions was not statistically significant from the assay buffer only incubations. Therefore, no significant DPP-IV activity was present in the rHSA solution.

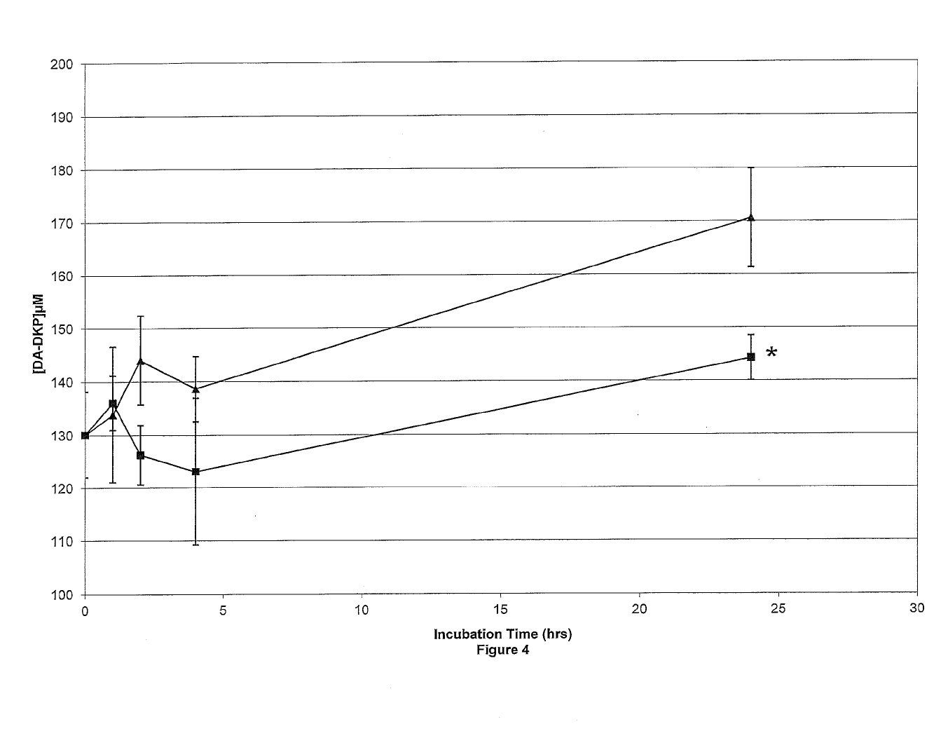

The formation of the DKP, DA-DKP, was measured in a commercial HSA solution heated at 60° C. in the presence or absence of a known DPP-IV inhibitor (diprotin A). The low molecular weight fraction of HSA containing DA-DKP was isolated using a 5 kDa MWCO spin column. The <5 kDa fraction was assayed for DA-DKP content by LCMS using negative electrospray ionization (−ESI). During the first 24 hours, DA-DKP content in the incubations containing no inhibitor increased 30% from baseline DA-DKP levels (FIG. 4). In the presence of the DPP-IV inhibitor, only a 10% increase in DA-DKP production was observed over 24 hours at 60° C.

Administration of commercial human serum albumin (HSA) is potentially indicated in patients such as multi-trauma patients. Due to its heterogeneous nature, other components can contribute to the therapeutic effect of commercial HSA, such as proteases. One such protease, dipeptidyl peptidase IV (DPP-IV), can release a known immunomodulatory molecule from the N-terminus of albumin, aspartate-alanine diketopiperazine (DA-DKP). Commercial HSA solutions prepared, e.g., by Cohn fractionation were assayed for DPP-IV activity with a specific DPP-IV substrate and inhibitor. DPP-IV activity was assayed at 37° C. and 60° C. since commercial HSA solutions are pasteurized at 60° C. for 10-11 hours. DPP-IV activity in commercial HSA solutions was compared to other sources of albumin such as a recombinant albumin. Significant levels of DPP-IV activity were present in commercial HSA solutions. This activity was abolished using a specific DPP-IV inhibitor suggesting that DPP-IV activity is present in commercial HSA. This activity was also present at 60° C. with 70-80% activity remaining from the 37° C. incubate. No DPP-IV activity was present in the recombinant source suggesting that DPP-IV activity is only present in albumin solutions produced using the Cohn fractionation process. Finally, increases in the formation of DA-DKP were observed when HSA solutions were heated at 60° C. This formation was significantly decreased in the presence of the DPP-IV inhibitor. DPP-IV activity in HSA could result in the production of many by-products for the critically ill patient including DA-DKP.

### Example 2

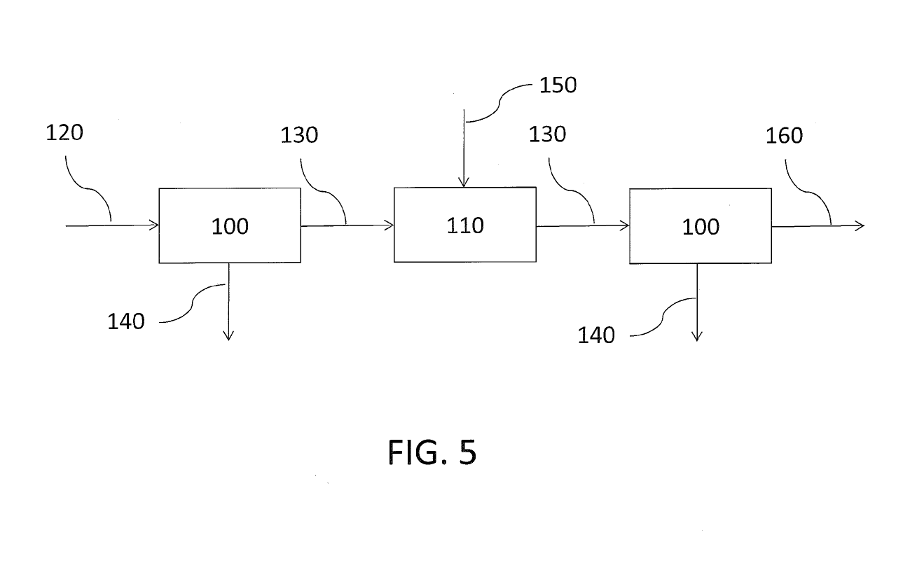

Referring first to FIG. 5, one embodiment of the present disclosure is shown in block diagram format, a method for treating a feed stream 120 comprising albumin and optionally DKP to produce therapeutic compositions. The feed stream 120 can comprise, for example, a saline solution comprising about 25 wt. % human serum albumin produced by the Cohn process and containing aspartic acid-alanine diketopiperazine (DA-DKP) in concentrations ranging from about 50 μM DA-DKP to about 100 μM DA-DKP on an albumin-free basis. The feed stream can also comprise sodium acetyltryptophanate, N-acetyltryptophan, and sodium caprylate, of varying concentrations. The feed stream is fed to a first processing step 100, comprising for example, tangential flow filtration which provides a size exclusion separation, wherein any molecules with less than from about 66 to about 69 kDa molecular weight pass through the filter in a first albumin-lean stream 140 (the filtrate). In this example, the first albumin-lean stream comprises essentially no albumin; ˜0 wt. % albumin. In other words, about 100% of the albumin in the feed stream 120 is retained in the first albumin-rich stream 130. The first albumin-lean stream 140 comprises a saline solution with DA-DKP concentrations ranging from about 50 μM DA-DKP to about 100 μM DA-DKP, on an albumin-free basis. The retentate retains any molecules with molecular weights greater than from about 66 to about 69 kDa, in a first albumin-rich stream 130, as well as any DKP-containing saline solution that is not forced through the tangential flow filter.

In this example, a theoretical maximum amount of DA-DKP is present in the feed stream 120, either as free molecules present as the product of thermal, chemical, and/or enzymatic degradation of the N-terminal and/or C-terminal ends, or successive ends, of albumin, or as unreacted albumin.

Referring to FIG. 5, the first albumin-rich stream 130 is then fed to a reacting step 110. The reacting step can comprise a temperature and pH controlled reactor, for example a stirred tank reactor or vessel similar to a fermentation vessel. In this example, an enzyme 150 is present in, produced in and/or metered into a heated reactor that is maintained at about 50° C. and maintained at a pH of about 5.0 by the addition of dilute sulfuric acid (not shown). In this particular example, the enzyme added 150 comprises dipeptidyl peptidase IV. Sufficient dipeptidyl peptidase IV (DPP-IV) is added to the reacting step 110 to provide peptidase activity from about 40 μM pNA to about 150 μM pNA. The reacting step 110 in this example is a batch reactor, wherein the reactants, albumin and DPP-IV, are maintained in the reactor at the set-point temperature and pH from about one hour to about 24 hours. The resultant reaction stream, the second albumin-rich stream 130, is subsequently processed in a second processing step 100.

In this particular example, the reacting step produces a significant amount of additional DA-DKP by the enzymatic degradation of the N-terminal and/or C-terminal ends, or successive ends, of the albumin. This can result in an increase in the concentration of DA-DKP in the second albumin-rich stream 130, on an albumin-free basis. So whereas the feed stream 120 DA-DKP concentration can have ranged from about 50 μM DKP to about 100 μM DA-DKP on an albumin-free basis, the second albumin-rich stream 130 DA-DKP concentration can range from about 100 μM DKP to about 150 μM DA-DKP on an albumin-free basis.

The second albumin-rich stream 130 is fed to a second processing step 100. In this example, the second processing step 100 is a second independent unit operation. Thus, it can be a second tangential flow filtration unit, or some altogether different technology; e.g., chromatography column. Alternatively, the second processing step could be accomplished by using the same equipment that was used in the first processing step, for example, by running the process in batch or semi-batch mode. In this example, the second processing step 100 is a second dedicated tangential flow filtration unit that operates on the same principles as the first unit described above in this Example 2.

In this Example 2, as described above, the second albumin-rich stream 130 contains a higher DA-DKP concentration than the feed stream 120. However, there is less albumin-free saline solution present due to the saline that was removed during the first processing step 100. Thus, the incremental gain in yield of the theoretical amount of DA-DKP in this Example 2 is inherently greater.

The filtration of the second albumin-rich stream 130 results in a final albumin-rich product stream 160, a first therapeutic composition, and a second albumin-lean (albumin-free in this case) stream 140, comprising a saline solution with DA-DKP concentrations ranging from about 100 μM DKP to about 150 μM DA-DKP on an albumin-free basis. The first and second albumin-lean DA-DKP-containing streams can be combined into one stream, forming the second therapeutic composition. For example, the albumin-rich product stream 160 can be used to treat conditions such as, but not limited to, malnutrition, starvation, nephrotic syndrome, pancreatitis and peritonitis. The combined DA-DKP containing albumin-free stream can then be used to treat human autoimmune disorders.

Although FIG. 5 illustrates only one reacting step 110 and only two processing steps 100, this is not intended to limit the scope of the present disclosure to one reacting step and two processing steps. Additional reacting and processing steps can further increase the DA-DKP yield. For example, a cumulative yield could be achieved after three reacting steps 110 and four processing steps 100. One of ordinary skill in the art will understand that the number of processing and reacting steps, and their arrangements relative to one another (e.g., in series, in parallel, with recycle loops, without recycle loops, etc.) will depend upon a comprehensive economic analysis that will vary from site-to-site and from application-to-application.

### Example 3

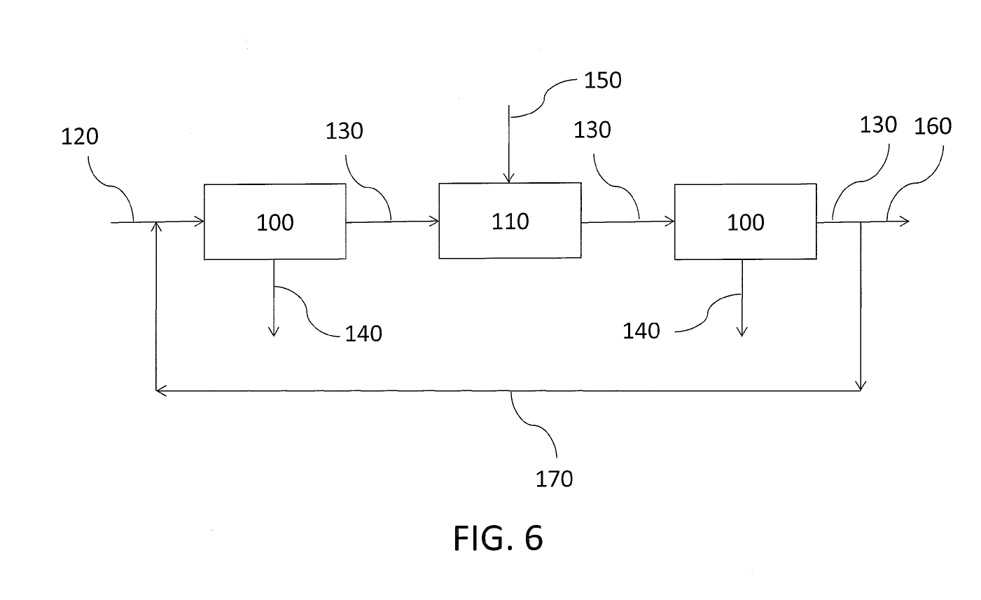

Referring now to FIG. 6, a variation of Example 2 is illustrated in block-diagram format, a method for treating a feed stream 120 comprising albumin and optionally DKP to produce therapeutic compositions, further comprising an albumin-rich recycle stream 170.

This example also comprises two processing steps 100 and one reacting step 110. In this example, a case is assumed wherein the DKP yield after these steps is unacceptably low; e.g., less than 50%. Thus, the albumin-rich stream 130 exiting the second processing step 100 is split into an albumin-rich recycle stream 170 which is recycled back to be combined with the feed stream 120 before it is fed to the first processing step 100, to give the albumin a second pass through the system to increase the yield above 50%.

In this example, it is envisioned that the process is run in continuous mode. Therefore, a final albumin-rich product stream 160 is continuously removed from the process, while fresh feed material 120 is continuously fed into the process. The internal recycle loop 170 can be significantly larger than the feed stream 120 and stream 160, with the actual magnitudes and ratios of these streams depending upon the per pass yields obtained in the processing steps 100.

### Example 4

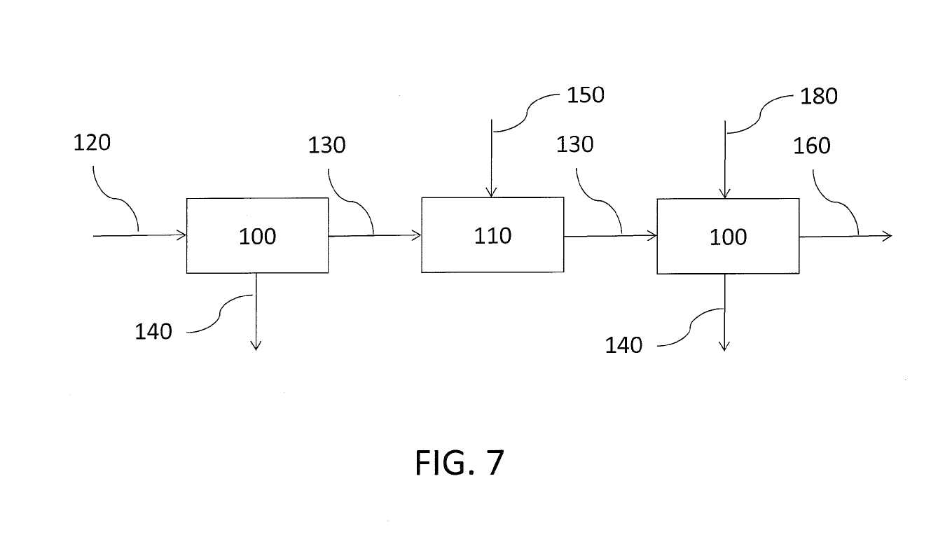

Referring now to FIG. 7, one further embodiment is illustrated of a method for treating a feed stream 120 comprising albumin and optionally DKP to produce therapeutic compositions, wherein Example 2 is modified to include a diluent stream 180 fed to the second processing step 100.

This example envisions the need to provide a displacement fluid that will displace the DKP-containing aqueous phase from the albumin during the processing steps. In this example, a Lactated Ringer's solution is used as a diluent stream 180 to displace more of the DKP present in the aqueous phase through a tangential flow filter unit.

The invention illustratively disclosed herein suitably may be practiced in the absence of any element, which is not specifically disclosed herein. It is apparent to those skilled in the art, however, that many changes, variations, modifications, other uses, and applications to the method are possible, and also changes, variations, modifications, other uses, and applications which do not depart from the spirit and scope of the invention are deemed to be covered by the invention, which is limited only by the claims which follow.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. The features of the embodiments of the invention may be combined in alternate embodiments other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations, combinations, and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating a feed stream comprising albumin and aspartic acid-alanine diketopiperazine (DA-DKP) to produce compositions, the method comprising:
processing the feed stream to produce a first albumin-lean stream and a first albumin-rich stream, wherein the first albumin-lean stream comprises a first portion of the DA-DKP present in the feed stream, and the first albumin-rich stream comprises a second portion of the DA-DKP present in the feed stream;
reacting the first albumin-rich stream in order to produce DA-DKP resulting in a reaction stream comprising albumin and DA-DKP; and
processing the reaction stream to produce a second albumin-lean stream and a second albumin-rich stream, wherein the second albumin-lean stream comprises a portion of the DA-DKP present in the reaction stream, and the second albumin-rich stream comprises a second portion of the DA-DKP present in the reaction stream.

2. The method of claim 1, wherein at least one of the first and second albumin-lean streams possess therapeutic value.

3. The method of claim 1, wherein at least one of the first and second albumin-rich streams possess therapeutic value.

4. The method of claim 1, wherein processing at least one of the feed stream and the reaction stream comprises at least one of filtration, chromatography, precipitation, extraction, and combinations thereof.

5. (canceled)

6. (canceled)

7. The method of claim 1, wherein reacting comprises at least one of heat treating, chemically reacting, enzymatically reacting, and combinations thereof.

8. The method of claim 7, wherein reacting comprises heating the first albumin-rich stream to an average bulk temperature ranging from about 40° C. to about 80° C.

9. The method of claim 7, wherein reacting comprises enzymatically reacting the first albumin-rich stream with at least one dipeptidase, kallikrein, cathepsin, carboxypeptidase, and combinations thereof.

10. (canceled)

11. (canceled)

12. The method of claim 1, wherein reacting comprises heating the first albumin-rich stream to an average bulk temperature ranging from about 40° C. to about 80° C. in the presence of dipeptidyl peptidase IV.

13. (canceled)

14. The method of claim 1, wherein the feed stream comprises at least one additional component selected from the group consisting of sodium acetyltryptophanate, N-acetyltryptophan, sodium caprylate, caprylic acid and combinations thereof.

15. The method of claim 1, wherein the DA-DKP is selected from soluble DA-DKP, a DA-DKP salt, and combinations thereof.

16.-19. (canceled)

20. The method of claim 1, wherein the first albumin-lean stream comprises DA-DKP concentrations of at least about 50 μM.

21. The method of claim 1, wherein the second albumin-lean stream comprises DA-DKP concentrations of at least about 50 μM.

22. The method of claim 1, further comprising an analyzing step, wherein the analyzing step comprises:
analyzing the second albumin-rich stream to yield at least one metric; and
comparing the at least one metric to at least one reference value, wherein when the at least one metric is less than the reference value, the reacting and processing steps are repeated until the at least one metric of a subsequent albumin-rich stream is equal to or greater than the at least one reference value.

23.-29. (canceled)

30. A composition comprising DA-DKP in a concentration greater than about 100 μM.

31. The composition of claim 30, wherein the concentration of albumin is less than 1 weight percent.

32.-33. (canceled)

34. The composition of claim 30, wherein the composition is prepared from a human serum albumin composition.

35. (canceled)

36. The composition of claim 34, wherein the preparation comprises enzymatic conversion of albumin to produce DA-DKP.

37. (canceled)

38. The composition of claim 30, further comprising at least one additional component selected from the group consisting of saline, Lactated Ringer's solution, Ringer's acetate solution, hydroxyethyl starch solution, dextrose solutions, and combinations thereof.

39. The composition of claim 30, further comprising at least one additional component selected from the group consisting of sodium acetyltryptophanate, N-acetyltryptophan, sodium caprylate, caprylic acid and combinations thereof.

40. A method of making a composition containing DA-DKP, comprising:
prior to filtering a plasma, performing:
a) contacting an unfiltered plasma with an enzyme that cleaves an N-terminal dipeptide from a protein in the plasma, and
b) heating the plasma under conditions effective to cause the formation of DA-DKP, or a physiologically-acceptable salt thereof.

41. The method of claim 40 wherein the protein is albumin.

42. The method of claim 40, wherein the step of heating is conducted at a temperature from about 40° C. to about 80° C.

43. The method of claim 40, wherein the enzyme comprises dipeptidyl peptidase IV.

44.-45. (canceled)

46. The method of claim 40, wherein the plasma further comprises at least one additional component selected from the group consisting of sodium acetyltryptophanate, N-acetyltryptophan, sodium caprylate, caprylic acid and combinations thereof.

47. (canceled)

48. A method of making a composition containing DA-DKP, comprising:
a) contacting an albumin-containing solution with an enzyme that cleaves a pair of N-terminal amino acids from the albumin, and
b) heating the albumin-containing solution under conditions effective to cause the formation of DA-DKP, or a physiologically-acceptable salt thereof.

49.-54. (canceled)
